(19) United States
(12) Patent Application Publication
(10) Pub. No.: US 2012/0215316 A1
(43) Pub. Date: Aug. 23, 2012

(54) EXPANDABLE INTERVERTEBRAL SPACER

(75) Inventors: Karen E. Mohr, Salt Lake City, UT (US); Jordan A. Hoof, Phoenix, AZ (US); Brad A. Niese, Chandler, AZ (US); Chad Lewis, Layton, UT (US); Jared M. White, Salt Lake City, UT (US)

(73) Assignee: MEDICINELODGE, INC. DBA IMDS CO-INNOVATION, Logan, UT (US)

(21) Appl. No.: 13/396,182

(22) Filed: Feb. 14, 2012

Related U.S. Application Data
(60) Provisional application No. 61/442,471, filed on Feb. 14, 2011.
(60) Provisional application No. 61/442,491, filed on Feb. 14, 2011.

(51) Int. Cl. A61F 2/44

(52) U.S. Cl. 623/17.16

(57) ABSTRACT

A spinal implant system, specifically an intervertebral spacer. The system is designed to change its physical conformation from a minimal profile to an expanded state, enabling it to be placed through a smaller incision and operative cannula. The ability to change from a minimal profile to an expanded state may be accomplished through pivoting of support bodies, or expansion through a screw system, or sliding of the support bodies perhaps along an oblong surface. The system will allow for long-term promotion of osteointegration.

### CROSS REFERENCE TO RELATED APPLICATIONS

This application claims the benefit of the following, which are incorporated herein by reference in their entirety:

Pending prior U.S. Provisional Patent Application No. 61/442,471 filed Feb. 14, 2011, which carries Applicants' docket no. MLI-92 PROV, and is entitled EXPANDABLE INTERVERTEBRAL SPACER WITH TURNBUCKLE LEAD SCREW; and

Pending prior U.S. Provisional Patent Application No. 61/442,491 filed Feb. 14, 2011, which carries Applicants' docket no. MLI-94 PROV, and is entitled EXPANDABLE INTERVERTEBRAL SPACER WITH SLIDING WEDGE.

### SUMMARY

Expandable intervertebral spacers and interbody devices are disclosed which are compatible with minimally invasive surgical techniques. Each disclosed device is designed to change its physical conformation from a minimal profile to an expanded state, enabling it to be placed through a smaller incision and operative cannula than other intervertebral spacers or interbody devices that do not undergo a corresponding change in conformation. The embodiments may share commonalities in their surgical approach and function. All disclosed devices are designed to function as passive intervertebral spacers. In many embodiments, the disclosed devices incorporate a cavity or central void to place bone graft, bone cement, or other structural and/or therapeutic material. The disclosed devices may promote long term osseointegration and fusion of the device into a bony construct suitable for supporting vertebral loads. All disclosed devices include one or more elements designed for long term implantation and compressive load bearing and transfer. Any of the devices disclosed herein may be suited for use in conjunction with supplemental vertebral fixation, such as certain plating or screw systems. A variety of methods and approaches may be used to place the devices. For example, devices may be implanted through a minimally sized incision. An operative cannula may optionally be used. Furthermore, no one particular embodiment is preferred to another, rather, each disclosed embodiment is a standalone alternative to achieving intervertebral fixation with an expandable device.

### BRIEF DESCRIPTION OF THE DRAWINGS

Various embodiments of the present system will now be discussed with reference to the appended drawings. It is appreciated that these drawings depict only typical examples of the present system and are therefore not to be considered limiting of the scope of the invention, as set forth in the appended claims.

FIG. 1A is a cephalad view of a compressed expandable intervertebral spacer centrally positioned relative to an adjacent vertebral body;

FIG. 1B is a cephalad view of the expanded expandable intervertebral spacer of FIG. 1a centrally positioned relative to an adjacent vertebral body;

FIG. 2A is a cephalad view of another compressed expandable intervertebral spacer centrally positioned relative to an adjacent vertebral body;

FIG. 2B is a cephalad view of the expanded expandable intervertebral spacer of FIG. 2a centrally positioned relative to an adjacent vertebral body;

FIG. 3 is a lateral view of another expandable intervertebral spacer;

FIG. 4 is a cephalad view of the spacer of FIG. 3 and an expander tool;

FIG. 5 is a lateral view of yet another expandable intervertebral spacer;

FIG. 6 is a cephalad view of yet another expandable intervertebral spacer centrally positioned relative to an adjacent vertebral body, where the spacer is in a first configuration;

FIG. 7 is a cephalad view of the spacer of FIG. 6 in a second configuration;

FIG. 8A is a perspective detail view of a component of the spacer of FIG. 6, showing an internal feature with a smooth portion and a partially threaded portion;

FIG. 8B is a cross section view of a component of the spacer of FIG. 6, showing an internal feature with a smooth portion and a partially threaded portion;

FIG. 9 is a cephalad view of the spacer of FIG. 6 in a third configuration;

FIG. 10A is a cephalad view of yet another expandable intervertebral spacer, where the spacer is in a first configuration;

FIG. 10B is a side view of the expandable intervertebral spacer of FIG. 10a, where the spacer is in a first configuration;

FIG. 11 is a cephalad view of the spacer of FIG. 10a in a second configuration.

FIG. 12 is a side view of yet another expandable intervertebral spacer;

FIG. 13 is a side view of pyramid teeth which may be on any of the spacers in FIG. 1, 3 or 10a to stack and interlock implant levels;

FIG. 14 is a partially exploded cephalad view of an expandable intervertebral spacer.

FIG. 15 is a cephalad view of another expandable intervertebral spacer.

FIG. 16 is a lateral view of the spacer of FIG. 15 in a compact state.

FIG. 17 is a lateral view of yet another expandable intervertebral spacer in a compact state.

FIG. 18 is a lateral view of the spacer of FIG. 17 in an expanded state.

FIG. 19 is a lateral view of yet another expandable intervertebral spacer in a first state.

FIG. 20 is a lateral view of the spacer of FIG. 19 in a second state.

FIG. 21 is a lateral view of yet another expandable intervertebral spacer in a first state.

FIG. 22 is a lateral view of the spacer of FIG. 21 in a second state.

FIG. 23 is a detail view of a screw of the spacer of FIG. 21.

### DETAILED DESCRIPTION

The devices disclosed in this application may be compatible with minimally invasive surgical techniques. The disclosed devices change their physical conformation, or configuration, from a first configuration, which may present a minimal insertion profile, to a second configuration, which may present an expanded profile. The first profile may enable any of the disclosed devices to be placed through a smaller incision and/or operative cannula than other devices that do not undergo a conformational change from a minimal profile to an expanded profile. The disclosed embodiments share commonalities in their surgical approach and function. All disclosed devices are designed to function as passive intervertebral spacers. The devices may incorporate a cavity or central void in which to place bone graft, bone cement, or other structural or therapeutic material. The devices may promote long term osteointegration and fusion of the device into a bony construct suitable for sustaining vertebral loads. The devices may include one or more elements designed for long term implantation and compressive load bearing and/or load transfer. Any of the devices disclosed herein may be suited for use in conjunction with supplemental vertebral fixation, such as certain plating or screw systems. A variety of methods and approaches are contemplated for the disclosed devices. Furthermore, no one particular embodiment is preferred to another, rather, each disclosed embodiment is a standalone alternative to achieving intervertebral fixation with an expandable device.

In this specification, standard medical directional terms are employed with their ordinary and customary meanings. Superior means toward the head. Inferior means away from the head. Anterior means toward the front. Posterior means toward the back. Medial means toward the midline, or plane of bilateral symmetry, of the body. Lateral means away from the midline of the body. Proximal means toward the trunk of the body. Distal means away from the trunk. Cephalad means toward the head of the body. Caudal means toward the feet.

In this specification, standard vertebral anatomical terms are employed with their ordinary and customary meanings.

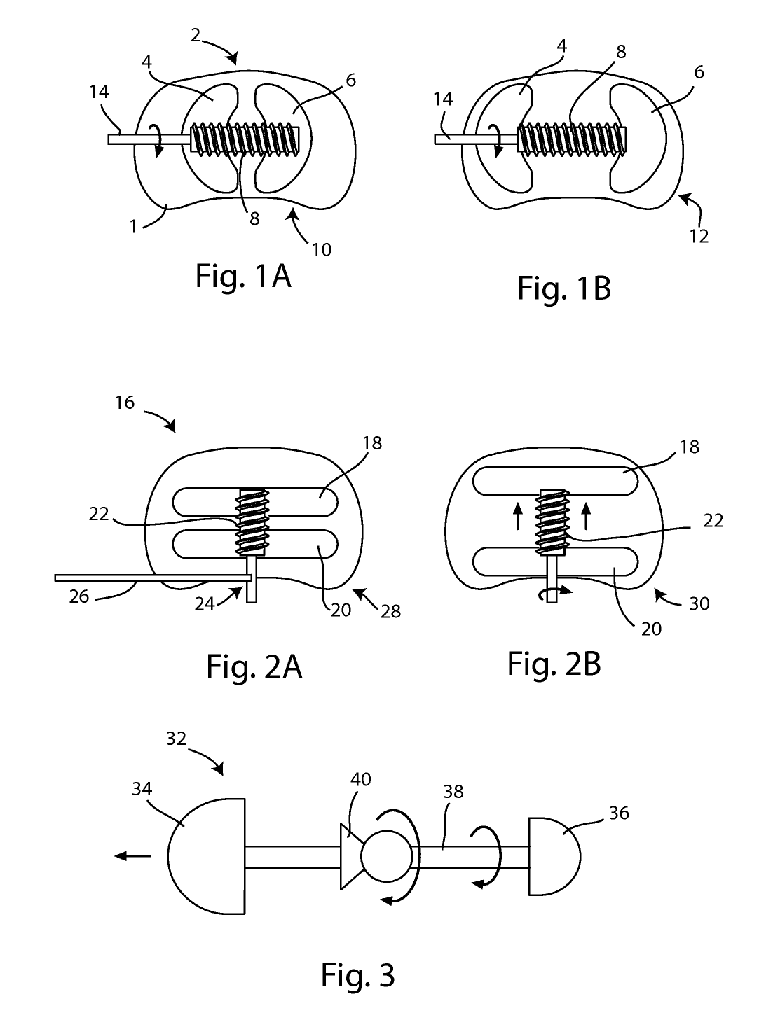

With reference to FIGS. 1a and 1b, an embodiment of an expandable intervertebral spacer 2 may include two intervertebral elements 4, 6 that are coupled to a deployment lead screw 8 between the vertebrae 1 and an adjacent vertebrae (not shown). The elements 4, 6 each include a body that may have a kidney-bean profile; the profile may also be circular, elliptical, polygonal, curved, or irregular. The deployment lead screw 8 can be mated to each element 4, 6 through a threaded interface. The deployment lead screw 8 may either be implantable or may be designed to be removed after deployment of the device 2 has occurred. Prior to deployment, the elements 4, 6 reside in a compact configuration 10 adjacent to one another, as is best seen in FIG. 1a. The lead screw 8 can run centrally through each element 4, 6 and be axially aligned in the direction of linear motion in which the elements 4, 6 deploy. The thread pitch as well as the number of thread leads on the lead screw 8 may be selected in order to control the amount of linear travel of each displaced element per rotation of the deployment lead screw 8. During deployment, the elements 4, 6 move away from each other, in order to provide the final expanded configuration 12 of the device, as is best seen in FIG. 1b.

Although it may not be perfectly depicted in the figures, the lead screw 8 may have opposing threads. On one side of the screw, the thread direction may run clockwise around the axis of the screw. On the other side of the screw 8, the thread direction may run counter-clockwise around the axis of the screw. Rotating the lead screw 8 in one direction may act to separate the elements 4, 6, while rotating the lead screw in an opposite direction may act to bring the elements 4, 6, closer together. Alternatively, one of the elements 4 or 6 may be captively retained and/or concentrically mated to the axis of the deployment mechanism, yet unengaged with the thread of the lead screw 8. The captive element may be proximally located nearer to a turning instrument 14 and/or a surgeon. A unidirectional thread would freely rotate through the stationary captive element and linearly translate the remaining element.

In a method of use, the device 2 may be deployed, or expanded, once the adjacent elements 4, 6 and threaded construct are appropriately positioned into the intervertebral space, between the vertebrae 1, by the surgeon. To deploy the device 2, an insertion tool may be removably coupled to the lead screw 8, for example by a hex socket and hex key arrangement. However, any polygonal socket or key arrangement may suffice. The device 2 may be positioned in the compact configuration 10, inserted into an intervertebral disc space from a lateral aspect of the vertebral body, advanced to a central location within the intervertebral disc space, and transformed to the expanded configuration 12 by turning screw 8 with instrument 14. Alternatively, the lead screw 8 may remain between the elements 4, 6 or be completely removed from the elements 4, 6 after transforming the device 2 into the expanded configuration 12, so that only the elements 4, 6 remain for long term implantation.

Referring to FIGS. 2a and 2b, another embodiment of an expanding intervertebral spacer 16 may include two intervertebral elements 18, 20 coupled to a lead screw 22. The elements 18, 20 each include a body that may resemble the elements 4, 6, and may, for example, have a more pronounced elongated shape than elements 4, 6. In spacer 16, the deployment interface 24 for turning the lead screw 22 can be angled relative to the axis of the lead screw 22. In one example, the interface 24 may make a 90 degree angle with the axis. The deployment mechanism or interface 24 of spacer 16 may be an angled gearbox with, for example, conical bevel gears. In other examples, the interface 24 may be a flexible shaft (not shown), universal joint, or ball hex to redirect the axis of deployment rotation to the axis of the lead screw 22. In this manner of angularly redirecting the rotation of the screw, the deployment mechanism or turning instrument 26 can be aligned with the more narrow summative width of the elements 18, 20 when in a compact configuration 28. Hence, in this arrangement, the deployment mechanism is no longer in line with the lead screw 22 or direction of travel, and the approach is more suitable for placement through a narrower cannula, as expansion of the elements 18, 20 occurs across the width of the cannula rather than along the length of the cannula. In a method of use, the device 16 may be positioned in the compact configuration 28, inserted into an intervertebral disc space from a lateral aspect of the vertebral body, advanced to a central location within the intervertebral disc space, and transformed to an expanded configuration 30 by turning screw 22 with instrument 26. Alternatively, the lead screw 22 may remain between the elements 18, 20 or be completely removed from the elements 18, 20 after transforming the device 16 into the expanded configuration 30, so that only the elements 18, 20 remain for long term implantation.

Spacers 2 and 16, in the examples shown, share a common expansion algorithm. For example, spacer 2 has a compact width in the minimal starting profile configuration 10, where the lead screw 8 is completely enclosed by the implant elements 4, 6. The maximum expansion of the elements of spacer 2 would be equal to the sum of the widths of each element along the screw. Therefore, the maximum footprint or expanded configuration 12 of the device 2 would be twice the initial profile width. Spacer 16 may operate similarly. In yet other examples the screw may “poke out” of the bodies to allow an even bigger footprint.

The elements of spacer 2 or 16 may be shaped to conform to the endplate geometry of the vertebral bodies—an individual element may have a superior-inferior projected profile representative of a “kidney bean” shape. Such geometry may be suitable for a spacer that expands in the medial to lateral direction, since the lateral aspects of the implant elements could be shaped to match the curvature of the lateral aspects of the vertebral endplate. Alternately, in an arrangement where a spacer expands in the posterior and anterior direction, the geometry of the element may be more suited to match the endplate geometry when the curvature of each element is “banana” shaped. The convex face of the elements would be oriented anteriorly while the concave faces would be facing posteriorly.

Referring to FIG. 3, yet another embodiment of an expanding intervertebral spacer 32 is shown in a partially expanded state. Spacer 32 may include two intervertebral elements 34, 36 and a lead screw 38. The elements 34, 36 of spacer 32 may have a semicircular or D-shaped profile in the lateral view as shown. In this embodiment, the lead screw 38 may include a ring gear 40 connected to the lead screw shaft. Ring gear 40 is shown centrally located between elements 34, 36 in the example shown in FIG. 3. However, other examples may have ring gears that are not centrally located.

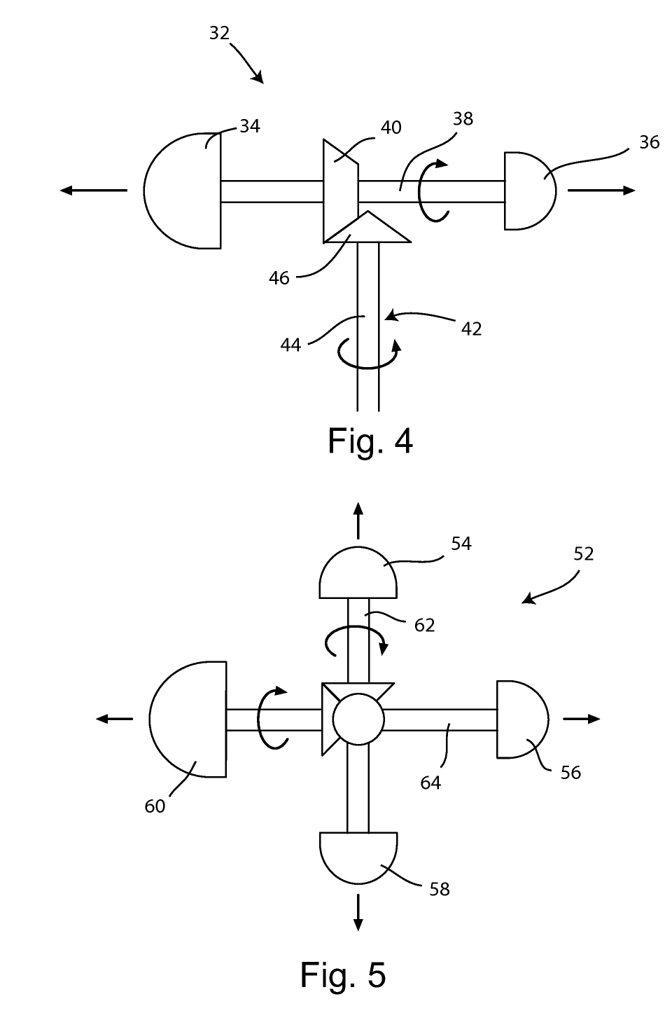

Referring to FIG. 4, spacer 32 is shown with a removable expander tool 42. Tool 42 may include a shaft 44 and a drive gear 46. Ring gear 40 and/or drive gear 46 may be bevel gears. Drive gear 46 meshes with ring gear 40 to transmit rotation from shaft 44 to lead screw 38. In the present arrangement, shaft 44 is shown generally perpendicular to screw 38, although parallel and oblique arrangements are contemplated. A portion of lead screw 38 may have right-hand threads, and another portion of lead screw 38 may have left-hand threads. Lead screw 38 may thread into each element 34, 36. For example, element 34 may have right-hand threads to engage the right-hand threaded portion of lead screw 38, and element 36 may have left-hand threads to engage the left-hand threaded portion of lead screw 38. In use, spacer 32 may be fitted tightly enough in an intervertebral disc space that elements 34, 36 are stabilized by adjoining vertebral endplates so that rotation of lead screw 38 results in pure translation of elements 34, 36 along screw 38. In another arrangement, spacer 2 may incorporate means for stabilizing elements 34, 36 against rotation due to the action of lead screw 38.

Referring to FIG. 5, yet another embodiment of an expanding intervertebral spacer 52 is shown in a partially expanded state. Spacer 52 may include four intervertebral elements 54, 56, 58, 60 and two lead screws 62, 64. In this embodiment, spacer 52 may provide simultaneous lateral and cephalad/caudal expansion in response to turning tool 42 (not shown).

A “dual axis” variation of the embodiments set forth above may utilize a plurality of lead screws, bevel gears and/or intervertebral elements for simultaneous expansion in two or more directions.

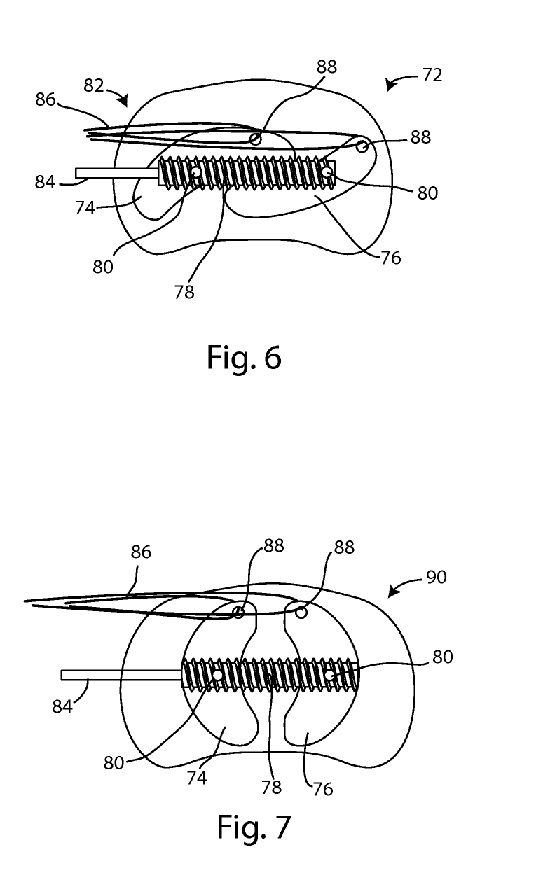

Referring to FIG. 6, yet another embodiment of an expandable intervertebral spacer 72 may include two intervertebral elements 74, 76 and a lead screw 78. In this embodiment, deployment rotation and lead screw rotation are co-axial. The elements 74, 76 may be coupled to the lead screw 78 with a pivoting and/or sliding attachment 80 so that the elements 74, 76 may be obliquely angled relative to the lead screw 78 in a compact configuration 82 of spacer 72. The elements may still be arranged immediately adjacent to one another, as described above, however the overall profile width of the initial pre-deployed configuration 82 may be smaller than the compact configuration 10 of spacer 2 because the individual elements 74, 76 are rotated and narrowly approximated in alignment to the direction of insertion, lead screw 78, and the deployment mechanism 84. On each element 74, 76, a suture or wire 86 can be affixed to a location 88 on the element radially distal to, or outwardly displaced from, the pivot point of the element. The location 88 may be a hole or aperture that allows passage of a suture or wire 86. The aperture 88 may be positioned toward one end of each element 74, 76 to allow for pivotal rotation of the elements 74, 76. The pivotal rotation may be in the transverse direction relative to the sliding attachment 80.

Referring to FIG. 7, in order to expand the intervertebral elements to an intermediate second configuration 90, the surgeon interfaces with the suture or wire 86 through the deployment mechanism 84, applying a load along the suture or wire 86 which is directed to the attachment point 88 located radially from the element pivot point 80, creating a torsional force and rotationally displacing the elements 74, 76 about their individual pivot points. In this manner, the elements' original position of approximated alignment with the lead screw is rotatably modified to the mid-deployment second configuration 90 in which the element length is perpendicular to the lead screw.

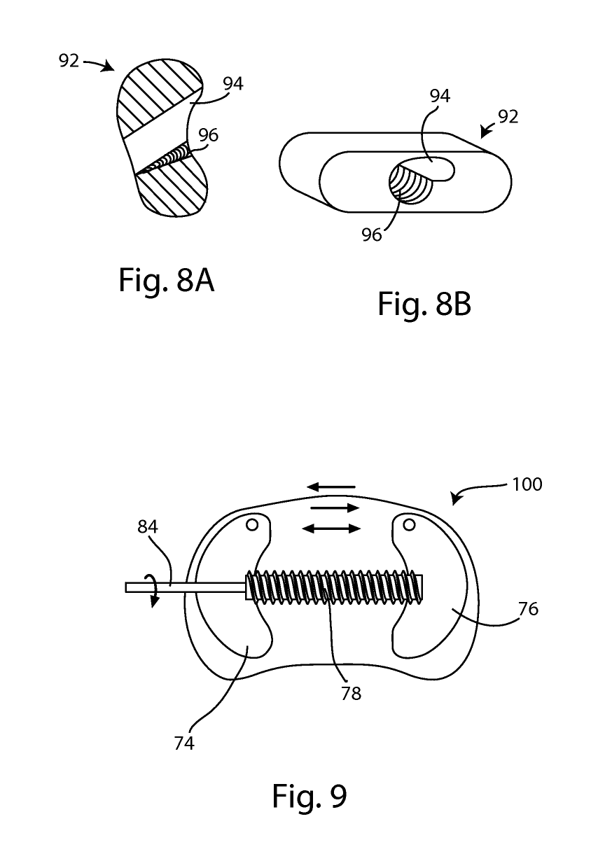

Referring to FIGS. 8a and 8b, the implant element 74, 76 may have an internal feature 92 to lock its rotational position. The internal feature 92, or bore, that may include a smooth channel 94 aligned with the lead screw 78 in the initial insertion orientation, and an offset threaded portion 96 that contacts the deployment lead screw 78 after the element 74, 76 rotates to its mid-deployment state.

Referring to FIG. 9, once the threaded internal portion 96 of the element 74, 76 is in contact with the lead screw 78, the deployment inserter 84 (or deployment mechanism), coupled to the lead screw 78, is rotated, driving the implant elements 74, 76 further apart and expanding the implant 72 to its final deployed configuration 100. The suture or wire 86 can be removed from the implant elements 74, 76 after deployment. The deployment inserter 84 may also be removed once the implant is in its final desired location. The lead screw 78 may remain between the elements 74, 76 but may also be removed once the implant 72 is in its final location.

In another example, a non-threaded implant element 74 or 76 may remain fixed at one end of the drive screw 78, while the other, internally threaded element is translated during drive screw rotation. The lead screw 78 may vary in length to accommodate different implant size profiles. The lead screw 78 may have a spherical feature to allow pivot of the elements 74, 76 and may function as a mechanical stop in order to promote retention of the implant element 74, 76 on the screw 78.

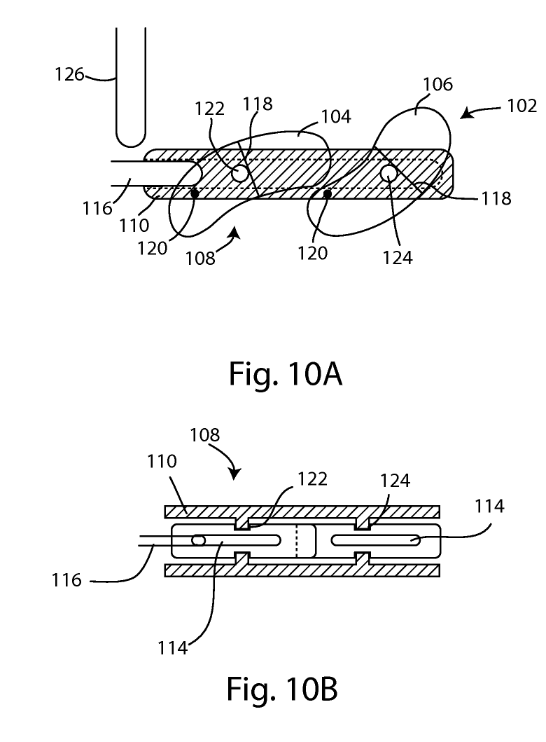

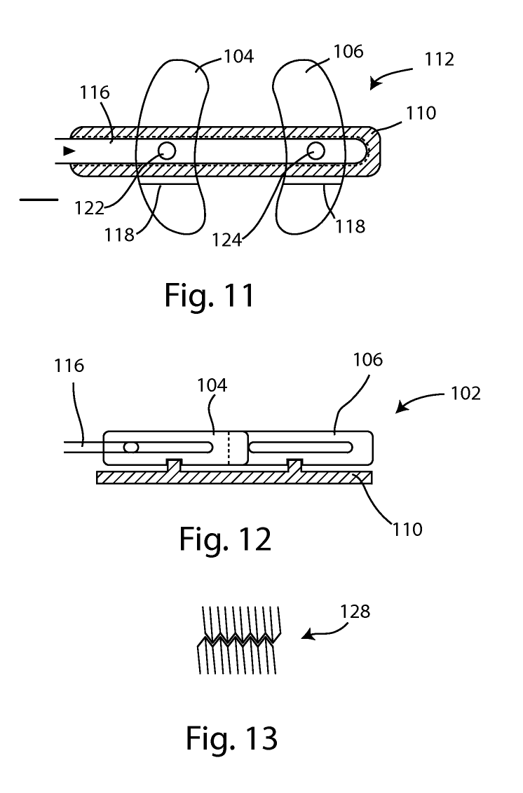

Referring to FIGS. 10a and 10b, yet another embodiment of an expandable intervertebral spacer 102 may include two intervertebral elements 104, 106. This embodiment has pivoting elements like spacer 72, but does not provide linear translation to further separate the implant elements 104, 106 after they are rotated from their compact conformation 108. Rather, the implant elements 104, 106 are pre-spaced on the inserter 110 to their final separation distance. The inserter 110 may include more than one plate on each side of the implant elements 104, 106. The elements 104, 106 are still deployed in rotation, changing the obliquely angled narrow alignment of each element in the compact configuration 108 to be perpendicular to the inserter 110 in an expanded configuration 112 (FIG. 11). However, rather than having a suture or wire 86 attached radially distal to the pivot point of each element, the elements 104, 106 may contain an internal through slot feature 114, which in the pre-deployed state 108, serves as a sliding ramp to cause rotation of the elements 104, 106. A rod 116, aligned with the length of the insertion device 110, is pushably movable having sufficient linear translation to run along the inserter 110 and through the central slots 114 of the elements 104, 106. The end of the push rod 116 may have a rounded or spherical tip to facilitate sliding along the sidewall ramp created by the through slot 114 in the implant element 104, 106. As the push rod 116 contacts and begins pushing on the misaligned ramp 118 created by the obliquely angled element's through slot 114, enough force is generated to disengage a small bump retaining feature 120 between the element and the inserter 110 which prevents the elements 104, 106 from rotating prematurely. The element 104, 106 rotates as the push rod 116 is inserted farther into the element's slot 114 until the face of the ramp 118 aligns with and is parallel with the outside diameter and axis of the push rod 116 (FIG. 11). At this point, the individual element 104 will have been rotated such that it is perpendicular to the axis of the inserter 110. As the push rod 116 continues its trajectory towards the second implant element 106, the disengagement from the retainer bump 120 and rotational displacement of the second element 106 is repeated in the same fashion. After the internal ramp surfaces 118 of both implantable elements 104, 106 has aligned with the push rod 116, the assembly 102 is free to be manipulated as required within the intervertebral space. Until the push rod 116 is removed, the elements 104, 106 are locked in perpendicular alignment to the inserter 110 by pivot points 122, 124 and the surface of the ramp contacting the push rod 116. The pivot points 122, 124 may be bosses projecting from each plate of the inserter 110 into the bodies of the implant elements 104, 106. The implant element pivot points 122, 124 may be recesses within the bodies of the implant elements 104, 106 configured to receive the bosses. Ultimately, the superior to inferior compression on the implants from the adjacent vertebral bodies and the engagement of the endplate bone into teeth on the superior and inferior surface of the implantable elements 104, 106 causes the position of the implants to be fixed within the intervertebral space. The deployment inserter 110 can be affixed to each rotating element at their respective pivot points 122, 124.

One method of engagement between the pivot point and the element may be a freely rotating locking detent mechanism. A ball, or another retaining feature, may be normally held within a cylindrical recess in the implant elements 104, 106, locking the element onto the pivoting point 122, 124 but still allowing free rotation. To release the elements 104, 106 from the deployment inserter 110, the mechanism holding the ball inside the recess is released, causing the ball to drop and the inserter 110 to disengage. In this manner, the insertion tool could be removed from the operative site while leaving the implant elements 104, 106 in their properly positioned alignment. The slots 114 in the implantable elements provide a convenient means for compacting bone graft material within the intervertebral space after deployment has occurred. Spacer 102 provides no-hassle positioning and a pre-spaced configuration that is highly repeatable.

FIG. 12 shows another embodiment of the spacer 102 with the deployment inserter 110 having a single plate positioned on one side, rather than both sides, of the implant elements 104, 106. The one plate or one side position of the inserter 110 may allow for easier removal of the inserter.

Another example of this design utilizes a flexible push rod 126 (FIG. 10) which is aligned at an angle to the main body of the inserter 110 on which the elements 104, 106 pivot. A flexible push rod 126 may provide better access to certain anatomies or spinal levels, such as L5-S1. Alternatively, instead of using a push rod that translates in pushing and pulling, a threaded rod could be engaged with the inserter 110, and as the threaded rodis rotationally deployed, it would translate linearly within the inserter body, through the element slots, into the ramp, causing the elements to rotate in the same fashion as with a push-pull rod.

Since the method of placing the elements 104, 106 of spacer 102 is highly repeatable, it may be possibly to easily stack multiple spacers in the cephalad—caudal direction and have the spacers interlock to provide an additional height increase to the intervertebral space. For example, the stacking feature could be a recessed shelf and lip on the superior side of a deployed implant, and another implant, having a complementary feature-mated boss on the inferior side, would be placed on top of the existing implant.

Referring to FIG. 13, another means to stack and interlock implant levels could be to use mated interlocking pyramidal teeth 128 on the implant faces. The pyramidal teeth 128 would restrict planar motion in two degrees of freedom as the adjacent pyramid features would be aligned such that they would otherwise interfere along the surface of a mated portion of another implant.

Some attributes which may be common to the above described embodiments found in FIGS. 1 through 13 may include, a narrow starting profile or compacted state, a pivoting design to change from compact state to expanded state, a stackable design for increased axial height in the cephalad-caudal direction, an expanding geometry that allows for narrower operative incision or cannula that may minimize patient morbidity, a turnbuckle lead screw embodiment that may be infinitely adjustable in lateral and/or anterior/posterior expansion planes to fit unique anatomy, and a high compressive strength with solid elements. Furthermore, the above described embodiments may include pivotable in-line element embodiments that may be conducive to a non-metallic construction, and pivotable in-line element embodiments that may have a highly repeatable surgical technique.

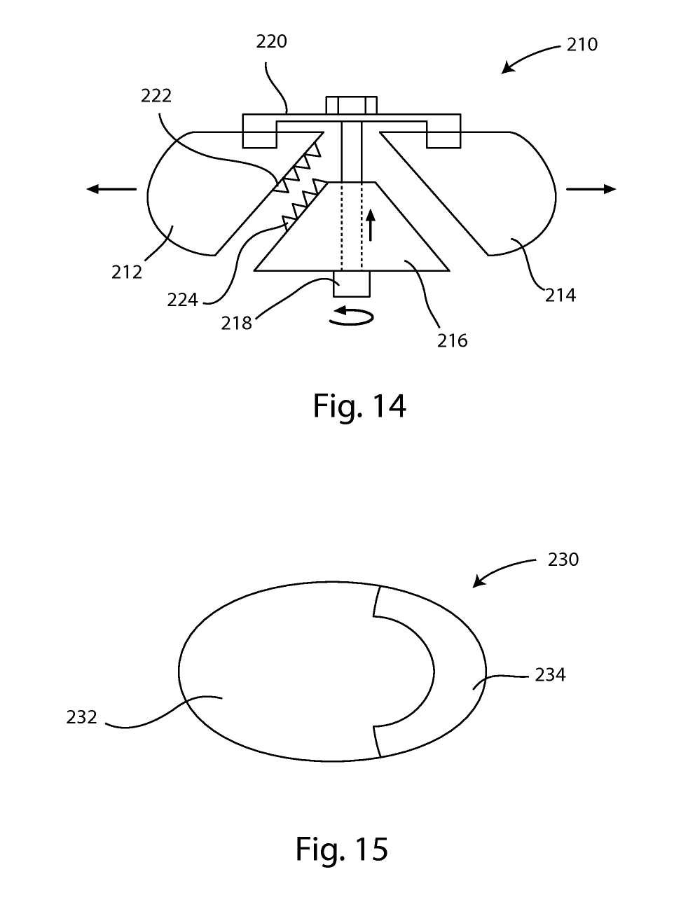

Referring to FIG. 14, an alternate embodiment of an expanding intervertebral spacer 210 may include a plurality of lateral portions 212, 214 and a wedge element 216. Lateral portions 212, 214 may also be described as wedge elements since they have oblique faces for interaction with the element 216. In the example shown in FIG. 14, wedge element 216 is centrally located between lateral portions 212, 214. Spacer 210 may also include a draw screw 218 and a housing or frame 220. Frame 220 may maintain the alignment and movement of lateral portions 212, 214 along a transverse axis of spacer 210. Frame 220 may also support and align screw 218 in order to maintain the alignment and movement of wedge element 216. Spacer 210 may have a compact state (not shown) in which the lateral portions 212, 214 are close to each other and wedge element is spaced apart from frame 220. Spacer 210 may have an expanded state (not shown) in which the lateral portions 212, 214 are spaced apart from each other and the wedge element 216 is close to frame 220. Spacer 210 may transform from the compact state to the expanded state in response to turning the screw 218. For example, screw 218 may slide through wedge element 216 and thread into frame 220. Turning the screw advances the screw through the frame and pushes wedge element 216 against the oblique faces of lateral portions 212, 214. In turn, lateral portions 212, 214 slide laterally outwardly away from wedge element 216. Spacer 210 may transform from the expanded state to the compact state in response to turning the screw 218 the other way. For example, a dovetail connection may be provided along the oblique faces of wedge element 216 and lateral portions 212, 214, wherein the dovetail is oriented to permit oblique sliding along the faces. The dovetail provides both tensile and compressive coupling of the wedge element 216 to the lateral portions 212, 214, so that the three components remain operatively engaged.

In the embodiment of FIG. 14, a plurality of intervertebral wedge elements 212, 216, 214 exist in a pre-deployed unexpanded state where the narrow most aspects of the individual wedges are initially aligned in contact. The interbody device is expanded by external actuation with instrumentation to push or pull the wedge elements 212, 216, 214 relative to one another, causing sliding along adjacent wedged faces of two or more wedge elements, shifting the initial alignment from the narrow wedge aspects to those aspects of greater width. As the narrow wedge aspects move apart, the wider aspects, along the wedge length in the direction of increasing slope, move closer together. For wedges of equivalent slope, the relative displacement of the wedge elements creates a total delta device expansion that is equal to the slope of the contacting wedge face multiplied by the total relative displacement distance of the wedges. The expansion in width of the device is equal to the wedge length plus the relative displacement of the wedges. The sloped wedge face may be oriented in any direction as simultaneous expansion is possible both axially and laterally.

Housing 220 may also be described as a supportive alignment fixture 220 that may be used to retain the wedges, in particular the lateral portions 212, 214 during deployment. Fixture 220 may also restrict the motion of the wedges 212, 214 such that the superior and inferior surfaces of the wedge elements remain parallel and aligned, thus limiting degrees of freedom in motion and only allowing translation in a relative sliding direction. Alternatively, the wedges could be interlocked together directly without a fixture 220 by creating a mated feature between the two sliding wedges, such as a tongue-and-groove or dovetail sliding lock. The shape and orientation of the wedge design may be such that deployment expansion occurs in the lateral, anterior-posterior, or the inferior-superior directions. For deployment to occur in the lateral direction, the sloped face of the wedge is directed laterally. Deployment along the height of the spinal column, or in the inferior-superior direction, would require the sloped face of the wedge element to be facing in either the superior or the inferior direction.

After deployment is complete and the appropriate level of expansion has been reached, the relative position of the blocks must be locked, limiting wedge sliding motion and retaining the implant expanded conformation. One possible way of achieving the lock would be to have directionally biased teeth 222, 224 appositionally mated with one another on contacting faces of the implant elements. The mating slopes of the teeth pairs would be such that sliding motion could only occur unidirectionally, allowing the wider aspects of the wedges to move closer together, in the manner in which expansion occurs, but not apart. The final relative position and maximum expansion of the wedge elements is initially locked and prevented from further expansion by the compressive load induced by the vertebral bodies pushing against the superior and inferior faces of the implant. Once boney fusion occurs, the relative position of the elements would be permanently locked together. Alternatively, a final locking feature or positive stop could be designed into the sliding faces of the implant elements to prevent further expansion of the device.

Wedge elements may be shaped such that the final footprint of the implant conforms to the native endplate anatomy in order to evenly distribute compressive loads along the fused column. Another option to increase accessibility would be to create smaller implants that conform to a portion of the endplate geometry, placing multiple implant sets and deploying them within the vertebral endplate area, further increasing the total contact area between the IBD devices and the bone and distributing the load among the deployed implants.

A variant on the sliding wedge design for expansion would utilize two expanding elements; not necessarily wedge shaped, and a cam mechanism. An instrument could be inserted into the device and would be used to provide a rotational displacement to a cam mechanism. A cam mechanism could be used for one or both of the following purposes—inducing two elements to separate and create device expansion OR locking the relative position of an expanded wedge device to maintain the expanded state of the device.

Referring to FIG. 15, another expanding intervertebral spacer 230 is shown in a compact state. Spacer 230 may include first and second support elements 232, 234 that may overlap in the caudal view as shown. The spacer 230 may be circular, elliptical or oval in a caudal view or may mirror the footprint between the intervertebral space (not shown). It will be appreciated that any shape that fits within the bounds of the intervertebral space may be applied including a polygonal or kidney bean shape.

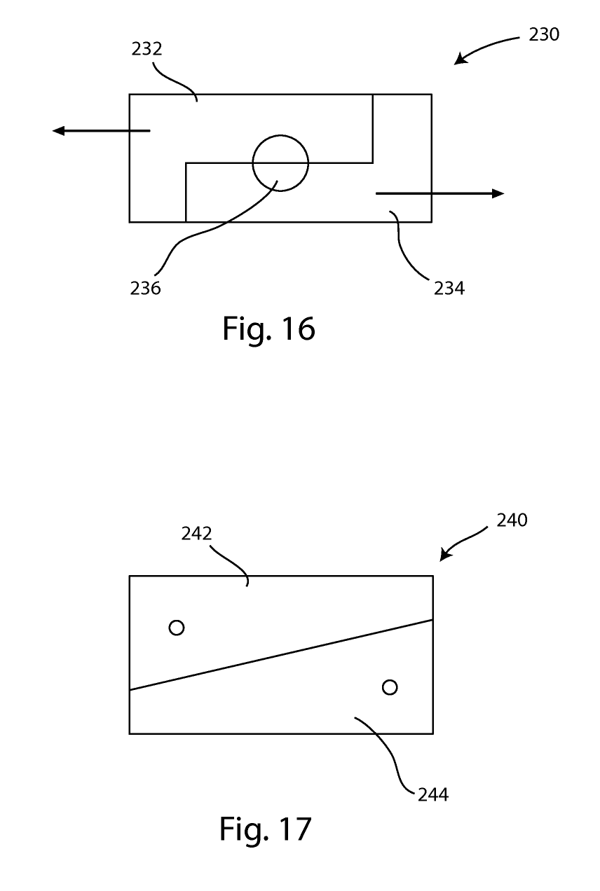

FIG. 16 shows spacer 230 in a lateral view in the compact state, and shows that a third component 236 may be included between elements 232, 234. Component 236 may actuate or deploy spacer 230 from the compact state to the expanded state, which is indicated with lateral arrows in FIG. 16. In the expanded state, elements 232, 234 slide laterally to increase the footprint of spacer 230. Component 236 may be, for example, a cam, winch, screw, or wedge that acts to cause relative sliding of elements 232, 234. The spacer, from a lateral view, may be rectangular and configured to provide adequate spacing in the intervertebral space. The height of the spacer may be as tall as the anatomic footprint of the intervertebral space.

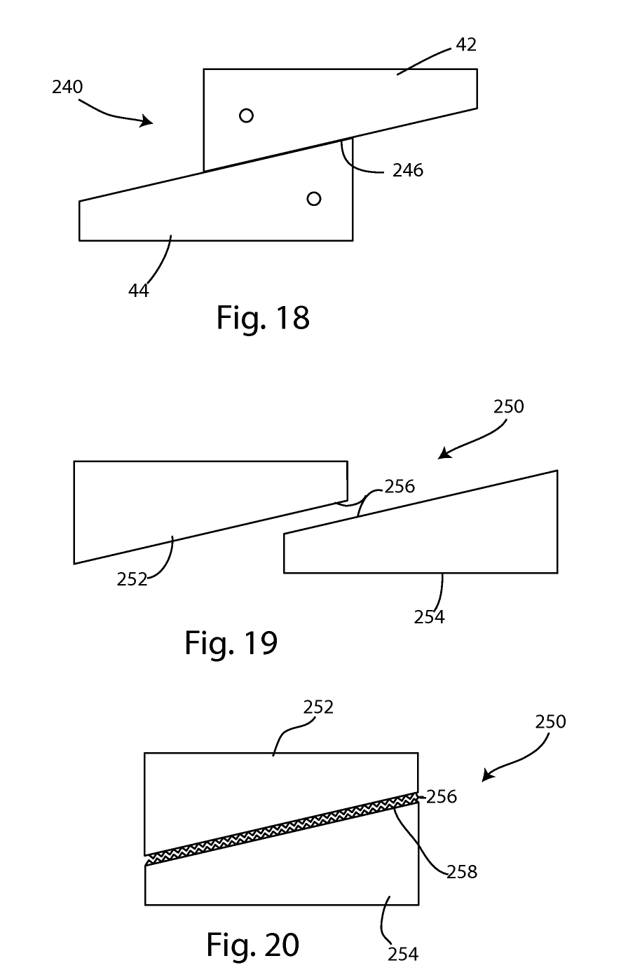

Referring to FIG. 17, yet another expanding intervertebral spacer 240 is shown in a lateral view in a compact state. The spacer 240 may have a profile in the cephalad/caudal view that resembles the profile of spacer 230 in FIG. 15. Alternatively, spacer 240 may have a round, polygonal, or kidney bean shape in order to fit as desired on a vertebral endplate. Spacer 240 may include a plurality of support elements 242, 244 that make contact along an oblique interface 246 so that elements 242, 244 may be described as wedge elements. FIGS. 17 and 18 show that lateral relative translation of elements 242, 244 results in lateral and vertical (cephalad-caudal) expansion of the spacer 40.

Referring to FIGS. 19 and 20, yet another expanding intervertebral spacer 250 is shown in a lateral view in an initial state (FIG. 19) and a final state (FIG. 20). Spacer 250 may include first and second support elements 252, 254 that make contact along an oblique interface 256. In this embodiment, spacer 250 may present a less stable configuration at first and a more stable, balanced final configuration. More specifically, elements 252, 254 may overlap slightly in the first configuration (FIG. 19) and may overlap significantly in the second final configuration (FIG. 20). The oblique surface 256 may be smooth or may include pyramidal teeth 258. The pyramidal teeth 258 may restrict planar motion between the elements 252, 254 and may aid in locking the elements together and preventing slip. The pyramidal teeth 258 may also provide a one way locking motion preventing the elements 252, 254 to revert back to an initial state (FIG. 19).

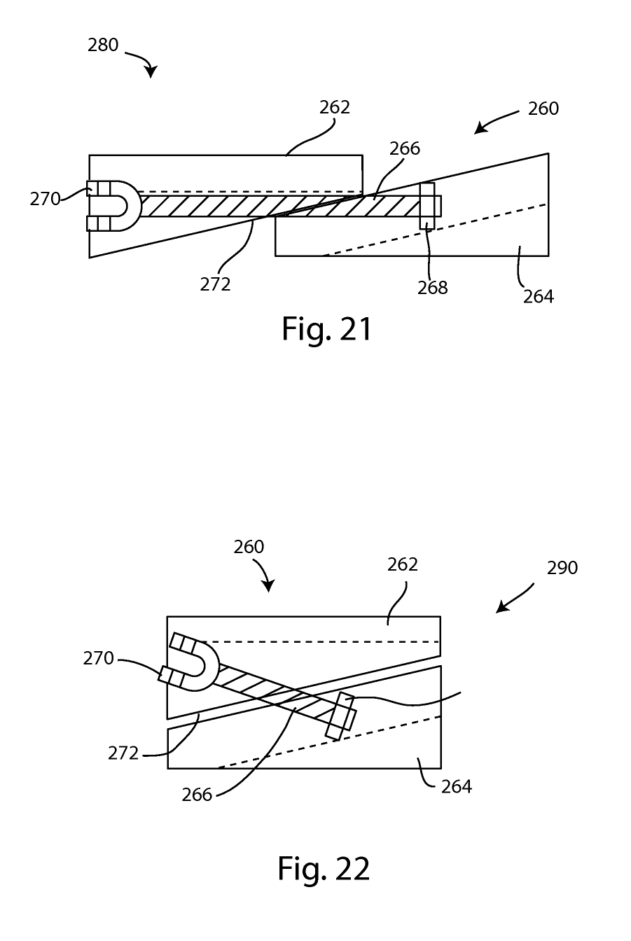

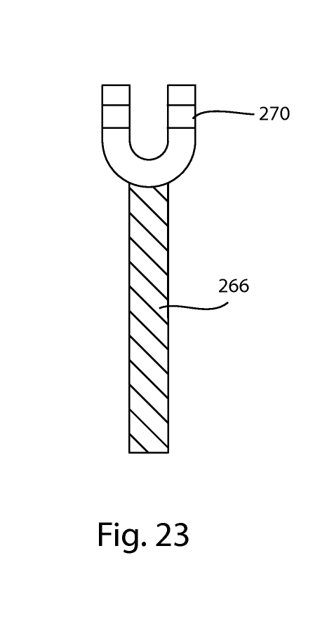

Referring to FIGS. 21-23, yet another expanding intervertebral spacer 260 is shown. Like spacer 250, spacer 260 may present a less stable initial configuration 280 and a more stable final configuration 290. Spacer 260 may include support elements 262, 264 each with bodies and a mobile draw screw 266 within the bodies of the support elements 262, 264. Screw 266 may have a universal joint head 270 toward one end of one support element 262, which is shown in more detail in FIG. 23. Head 270 may engage a complementary structure in element 262. The tip of screw 266 may engage a sliding translation nut 268 connected to element 264. The support elements 262, 264 may be mixed and matched such that the joint head 270 may be in element 264 and the nut may be in element 262. In this embodiment, the screw 266 may be generally parallel to an outside wall of the spacer 260 or parallel with the outside walls of the different support elements 262, 264 in its less stable 280 or smaller footprint configuration. The screw may then pivot and become oblique to the outside wall as screw 266 rotates to cause elements 262, 264 to slide relative to one another along a mutual oblique interface 272. The screw 266 may also pivot with respect to elements 262, 264 to increase the available travel in spacer 260.

A further variant and an alternative to external linear push/pull instrumentation actuation is the design in which a draw screw runs centrally through one or more of the deployment wedges. A threaded mechanism provides the potential of easy rotational deployment and locking the deployed state of the device. The screw freely rotates within the unthreaded proximal wedge element where the screw head resides. The screw is threaded distally into another wedge, or an alignment support frame containing and restricting the motion of other wedge elements. As the screw is rotated, the proximal wedge element is brought into proximity with another wedge element, causing the sloped faces of the wedge elements to contact and begin sliding against one another, initiating unidirectional expansion of the device. In one variant, two wedges are initially contacting at their narrow most aspects. A screw runs centrally through the wedge elements, linking them together and is free to pivot about the base of the screw head. A threaded nut holds the distal portion of the screw and remains captive within the second wedge element. The captive nut is free to rotate as well as translate within the second wedge body. As the screw is rotated for deployment, the captive nut is brought nearer to the head of the screw, drawing the second wedge element along the sliding face of the first wedge element. A universal joint configuration may be used to apply torque to and attach to the head of the screw, enabling the axis of the screw and trajectory of the nut's translation to freely change angle as it is deployed. In the final deployed conformation, the perimeters of the two wedge elements align evenly with one another so as to provide even support across the face of the vertebral bodies. The nut, having translated proximally towards the head of the screw provides compression against the internal captive nut slide feature of the second wedge element, firmly holding it in place against the first wedge element, and maintains the expanded conformation of the device.

Some attributes which may be common to the above described embodiments found in FIGS. 14 through 23 may include a sliding wedge expansion with fewer moving components or areas critical to strength; the ability to positively lock in a deployed state; expanding geometry allowing for narrower operative cannula and minimizes patient morbidity; designs conducive to non-metallic construction; the device may be designed to provide either axial or lateral expansion; narrower starting profiles; simplicity in their design and reliable and cost effective to manufacture; high compressive strength; repeatable results.

The present embodiments may be embodied in other specific forms without departing from its spirit or essential characteristics. It is appreciated that various features of the above described examples and embodiments may be mixed and matched to form a variety of other combinations and alternatives. As such, the described embodiments are to be considered in all respects only as illustrative and not restrictive. The scope of the described is, therefore, indicated by the appended claims rather than by the foregoing description. All changes which come within the meaning and range of equivalency of the claims are to be embraced within their scope.

1. A spinal implant system, comprising:
a plurality of implant elements; and
a structure, wherein each of the plurality of implant elements is operatively connected to the structure and each of the plurality of implant elements is displaced by the structure;
wherein the system comprises:
a compact configuration, wherein the system has a small footprint; and
an expanded configuration, wherein the system has a large footprint.

2. The system of claim 1, wherein, in the compact configuration, the plurality of implant elements are obliquely oriented to the structure; and
wherein, in the expanded configuration, the plurality of implant elements are orthogonally oriented to the structure.

3. The system of claim 2, wherein each of the plurality of implant element is pivotally connected to the structure.

4. The system of claim 3, wherein the plurality of implant elements each comprise a bore configured to receive the structure in a first slidable configuration and a second threadable configuration.

5. The system of claim 4, wherein the first slidable configuration is in the compact configuration and the second threadable configuration is in the expanded configuration.

6. The system of claim 5, wherein the plurality of implant elements each comprise an aperture toward one end of each of the plurality of implant elements, wherein the aperture is configured to receive a suture.

7. The system of claim 1, wherein, in the compact configuration, the plurality of implant elements are close to each other;
wherein, in the expanded configuration, the plurality of implant elements are far from each other.

8. The system of claim 1, wherein each of the plurality of implant elements is slidably connected to the structure.

9. The system of claim 1, wherein each of the plurality of implant elements is threadably connected to the structure.

10. The system of claim 1, wherein the structure comprises a threaded rod, wherein turning the threaded rod in a first direction transforms the system from the compact configuration to the expanded configuration.

11. A spinal implant system, comprising:
a plurality of implant elements wherein each of the plurality of implant elements comprises a body; and
a first plate, wherein each of the plurality of implant elements is pivotally connected to the plate;
wherein the system comprises:
a compact oblique configuration, wherein the system has a small footprint; and
an expanded orthogonal configuration, wherein the system has a large footprint.

12. The system of claim 11, wherein the system further comprises a push rod.

13. The system of claim 12, wherein each of the plurality of implant elements is pivotally engaged with the first plate.

14. The system of claim 13, wherein the plurality of implants elements further comprise a slot within the body configured to receive the push rod.

15. The system of claim 11, further comprising a second plate.

16. The system of claim 15, wherein the system further comprises a push rod wherein the push rode is positioned between the first plate and the second plate.

17. The system of claim 16, wherein the plurality of implant elements further comprise a slot within the body configured to receive the push rod.

18. A spinal implant system, comprising:
a plurality of support elements, wherein the plurality of support elements interact along an oblique interface therebetween;
wherein the system comprises
a compact configuration, wherein the system has a small footprint; and
an expanded configuration, wherein the system has a large footprint.

19. The system of claim 18, wherein the plurality of support elements comprise a pair of lateral support elements and a central support element, wherein the central support element is wedge shaped and engages both lateral support elements.

20. The system of claim 19, wherein, in the compact configuration, the lateral support elements are close together; and
in the expanded configuration, the lateral support elements are spaced apart.

21. The system of claim 18, wherein, in the compact configuration, the system has a first height, width, and depth; and
in the expanded configuration, the system has a second height, width, and depth;
wherein at least one of the second height, width, and depth is greater than the corresponding one of the first height, width, and depth.

22. The system of claim 18, further comprising a draw screw residing within bodies of the plurality of support elements wherein a head of the screw resides within the body of one of the plurality of support elements and a translation nut resides within the body of a separate one of the plurality of support elements.

23. The system of claim 22, wherein the draw screw resides parallel to an outside wall of the plurality of support elements in the compact configuration; and
wherein the draw screw resides oblique to the outside wall of the plurality of support elements in the expanded configuration.
